(19) United States
(12) Patent Application Publication
(10) Pub. No.: US 2011/0100380 A1
(43) Pub. Date: May 5, 2011

(54) Therapeutic footwear system, methods and devices

(76) Inventor: Mark Hampton, (US)

(21) Appl. No.: 12/612,906

(22) Filed: Nov. 5, 2009

(51) Int. Cl. A61B 19/00

(52) U.S. Cl. 128/898; 128/897

(57) ABSTRACT

The invention is a therapeutic system and its components, The system is composed of items of footwear, processes, methods, and apparatus of transformation of the items into therapeutic devices, and their application as therapeutic devices through application of bodyweight, and wear.

### CROSS-REFERENCE TO RELATED APPLICATIONS

20040216219

20050102734

20080301856

20050023288

6,931,768

569,557

5,584,077

4,734,938

4,728,538

11/858,149

### STATEMENT REGARDING FEDERALLY SPONSORED RESEARCH OR DEVELOPMENT

Not applicable

### REFERENCE TO SEQUENCE LISTING, A TABLE OR A COMPUTER PROGRAM LISTING COMPACT DISC APPENDIX

Not Applicable

### BACKGROUND OF THE INVENTION

Human beings throughout history have sought means to effect their environment. This fact has given rise to both science and spiritual practices. With respect to the latter, rituals and talismans are an important, integral part of the lives of many. Feng Shui is an eastern science or practice that involves the orientation and placement of objects toward the objective of stimulating or suppression forms of energy to influence circumstances of health, wealth, relationships, and the gamut of experiences. Like other forms of ritual, there are symbolic relationships between the objects and the actions, and the projection or desired outcome. The invention is the improvement and novel utility of specific objects toward a specific desired outcome.

It is human nature to experience or perceive adversarial influences. This may take the form of an individual, an organization, entity, energy, substance, etc. Without regard to placebo effect, there are a range of activities which are observed to be therapeutic in addressing these experiences. The invention is the improvement and novel utility of specific objects toward achieving a therapeutic effect.

The invention is utilized to provide therapeutic value to a person experiencing adversarial influences via the symbolism of putting the adversary “under one's feet”, to “walk all over them”. There are several colloquialisms related to this idea, but no prior art with respect to projection the notion into physical reality. The therapeutic effect may be psychological or metaphysical in nature.

### BRIEF SUMMARY OF THE INVENTION

The invention is a system of therapy, comprised of an item or component of footwear (hosiery, socks, insoles, shoes, heels, soles). containing a reference to an adversary, worn on the foot of the user, such that the reference to the adversary is consumed and/or destroyed. The invention includes the methods and apparatus used to transform the item or component of footwear from its generic state to a component of the system of therapy.

### BRIEF DESCRIPTION OF THE SEVERAL VIEWS OF THE DRAWING

Not Applicable-With all due respect to the examiner, the presumption is that footwear mentioned in the brief summary is a common experience, as are written words, photographs, etc.

### DETAILED DESCRIPTION OF THE INVENTION

As a system of therapy, the user is intent to take symbolic positioning and action with respect to an adversary. The system enables the users intent by presenting the mechanism through which to operate. The mechanism is created when a generic item of footwear is transformed via processes and apparatus claimed in this application. The therapeutic action occurs as the wearer's bodyweight is applied to the mechanism, and the mechanism wears. The wearing is apparent to the user, i.e., the effect of the process is effective observable in this regard.

### PERTINENT PRIOR ART

20040216219 Brevoort, Discloses Socks with permanent color labels. Brevoort is pertinent in that it refers to Marking items of footwear. However, its purpose as identification (color) and by the title of the application, its intent is to be permanent. Each claim in the referenced application refers to color name, and does not suggest that its claims transform the item nor manifest a new product nor purpose via the article's modification as does this application.

20050102734 Daniel et al, Discloses its purpose as to match individual socks with its mate, and for the purpose of identification. The referenced application does not suggest that a new item has been created via inclusion of a match indicator. This application does not claim as its purpose identification nor matching, making no reference to neither “pairs” nor “matches”.

Like Brevroot, Daniel et al seems to conflict directly with 4734938 Anderson, as the stated purpose of the marking is in context of identification. This application makes no such claim.

20080301856 Erringo-Sock Matching Apparatus and Method. Context similarly to Brevoort and Daniels et al, in that the current application makes no claim with respect to pairs, matching, nor association of any item footwear to another.

20020023288 Garneau-Athletic sock with printed strip. Apparently claims an existing item made from different material. The current application differs substantially.

6,931,768—Baek. The current application is similar to the patent issued to Baek in the a novel footwear item of specific purpose and structure is specified via a description of varying aspects of its composition. Sets precedent for the eligibility of the current application as a novel footwear item for a specific purpose as defined by elements of its composition

5699557 Johnson. is successful in specifying a purposeful apparel system manifest from existing components and technology, and based on inclusion of a decorative effect. Johnson is successful in light of 4734938 Anderson, even though the appliqué described by Johnson could, potentially, be used to identify the socks as in Anderson. Sets precedent for eligibility of the current application in light of Anderson, as footwear including components that could potentially be used for identification, but not so claimed.

4,734,938 Anderson ID socks-Anderson is successful in defining permanently marked socks for the purpose of matching. The current application differs in that the any markings are distinguished in purpose, and, are deliberately impermanent. Anderson is successful in light of 3,258,858 Cariiffe, even though the decoration described by Anderson could be used for training purpose as in Cariffe, and the training markings in Cariffe could be used for purposed of identification. These reinforce the precedent of eligibility of items of footwear, marked for a claimed purpose, that could potentially be used for other purposes.

3,258,858 Cariffe Successfully claims marking footwear items for a specific purpose distinct from 3,135,442 Carter, wherein marking footwear with its characteristics are successfully claimed. There is substantial precedent for successful claims of marking/decorating footwear as differentiated by purpose.

4,728,538 Kaspar Is successful in claiming the methods and apparatus to apply purposeful material to garments. The subject of this application is distinct However it is similar in claiming the methods and apparatus related to adding or modifying a garment. As the invention of this application is novel, and the methods are being claimed, novel apparatus used in those methods are likewise eligible via Kaspar.

5,584,077 Thrift is successful in claiming the methods for transforming an item of footwear into something that is experienced differently by the viewer, based on the viewers experience and knowledge, not otherwise materially altering the generic item; The current application is similar in this regard, while the subject and claims are distinct.

11/858,149 I, Mark Hampton, the current applicant, claim devices as specified in this application, but improperly, with respect to context and statutory matter. The substance and claims were disclosed, albeit improperly, in response to an office action of Oct. 15, 2009 in this regard.

1. A system/method of therapy consisting of:
a. An item of footwear
b. methods whereby the item in claim 1a is transformed into a therapeutic device.
c. Wearing of the therapeutic device on the foot
d. Transfer of the weight of the wearer onto therapeutic device
e. Consumption of the therapeutic transformation via wear.

2. An item in claim 1a where the item is a sock or other form of hosiery

3. An item in claim 1a where the item is an insole

4. An item in claim 1a where the item is an orthotic device

5. An item in claim a where the item is a shoe

6. An item in claim 1a where the item is a shoe sole or part thereof.

7. An item in claim 1a where the item is a shoe heel or part thereof.

8. An item in claim 1a where the item is a shoe coating or covering

9. An item in claim 1a where the item is a shoe sole coating or covering

10. An item in claim 1a where the item is a shoe heel coating or covering.

11. A method in claim 1b where the items in claim 1a are marked with reference(s) to an adversary(s).

12. Marking(s), as in claim 11, that is text

13. Marking(s), as in claim 11, that is an image

14. Marking(s), as in claim 11, that is a logo or symbol

15. Marking(s), as in claim 1, that is placed on or under the heel of the item.

16. Marking(s), as in claim 11, that is placed at on or under the arch of the foot of the item

17. Marking(s) as in claim 11, that is placed at the ball of the foot of the item

18. Marking(s) as in claim 11, that is placed at or under the toe of the foot of the item.

19. Marking(s) as in claim 11, that is placed under any combinations of claims 15-18

20. An adversary in claim 11, that is an entity

21. An adversary in claim 11, that is an health affect

22. An adversary in claim 11, that is an influence.

23. An adversary in claim 11, that is a AliAperson or group of people.

24. An adversary in claim 11, that is a substance

25. An adversary in claim 11, that is an emotion

26. An adversary in claim 11, that is an action

27. An adversary in claim 11, that is an experience

28. An adversary in claim 11, that is a place

29. An adversary in claim 11, that is an object

30. A method in claim 11, that is printing

31. A method in claim 11, that is direct to garment printing

32. A method in claim 11, that is pad printing

33. A method in claim 11, that is screen printing

34. A method in claim 11, that is thermal transfer

35. A method in claim 11, that includes a layer of material of any composition, adhered to the item in 1a.

36. A method in claim 11, that includes layer of material of any composition, marked and adhered to the item in claim 1a.

37. A method in claim 11, that is embossing

38. A method in claim 11, that is etching

39. A method in claim 11, that is burning

40. A method in claim 11, that is embroidery

41. A method in claim 11, that is weaving

42. A method in claim 11, that is the application of a solvent to remove coloring

43. A method in claim 11, that is manual, consisting of application of marking chemical or material.

44. A method in claim 11, that is moldeding/casting as a part of the item

45. Molds and casts as in claim 44.

46. Fixtures, used in claim 11 to position the item for marking, (automated or manual).

47. A method in claim 11 that Includes positioning the item in claim 1a onto or into a fixture in claim 44.

48. A method in claim 1b that includes positioning the fixture and/or item in claim 47 into a printing device.

49. Therapeutic devices as referenced in claim 1b-d,

50. The method of applying the wearer's bodyweight onto the Therapeutic device in claim 49.
